(19) United States
(12) Patent Application Publication
(10) Pub. No.: US 2004/0102440 A1
(43) Pub. Date: May 27, 2004

(54) Method of promoting smoking cessation

(76) Inventor: Erik Ho Fong Wong, Portage, MI (US)

Correspondence Address: PHARMACIA & UPJOHN, 301 HENRIETTA ST, 0228-32-LAW, KALAMAZOO, MI, 49007, US

(21) Appl. No.: 10/602,447

(22) Filed: Jun. 24, 2003

Related U.S. Application Data
(60) Provisional application No. 60/392,893, filed on Jul. 1, 2002.

(51) Int. Cl. A61K 31/537; A61K 31/4439

(52) U.S. Cl. 514/238.8; 514/343

(57) ABSTRACT

Disclosed is a method of promoting smoking cessation in a human comprising administration to the human an effective amount of reboxetine in combination with the administration of a smoking-cessation enhancing agent. Also disclosed is a composition for administration to a human for promoting smoking cessation comprising reboxetine and smoking-cessation enhancing agent. Examples of the smoking-cessation enhancing agents include nicotine, an antidepressant, a nicotine receptor antagonist, and an opioid antagonist.

### CROSS REFERENCE TO RELATED APPLICATIONS

This application claims the benefit of U.S. provisional application Serial No. 60/392,893, filed Jul. 1, 2002.

### BACKGROUND OF THE INVENTION

1. Field of the Invention

This invention relates to smoking cessation therapy and method, and specifically to use of reboxetine in combination with another smoking cessation agent in such therapy and method.

2. Description of the Related Art

Smoking continues to be a major health hazard in our society. It is thought to be the leading preventable cause of death in the United States, resulting in nearly 400,000 deaths per year due to smoking-related diseases such as cancer, heart diseases, and respiratory diseases. Moreover, smoking not only affects the health of a smoker, it may pose a health risk for non-smokers as well. Thus, smoking cessation is of great public interest.

Because of the grave consequences of smoking and other considerations, many smokers do desire to quit smoking. Quitting smoking, however, is exceedingly difficult. One survey shows that 74% of smokers report a desire to quit smoking and 70% of smokers have made previous attempts to quit smoking; yet success in quitting remains low. (Kolawole S. Okuyemi, MD, MPH; Jasjit S. Ahluwalia, MD, MPH, MS; Kari J. Harris, PhD, MPH. Pharmacotherapy of Smoking Cessation. Arch Fam Med. 2000; 9:270-281).

Many pharmacotherapies have been developed or explored for aiding smokers to cease smoking, the predominate one being nicotine replacement therapies. Nicotine replacement therapies involve the administration of nicotine through a suitable delivery system. Nicotine replacement products that are currently on the market includes (1) nicotine transdermal patches, such as NicoDerm® CQ® (GlaxoSmithKline), Habitrol® (Novartis Consumer Health), and Nicotrol® (Pharmacia Consumer Healthcare); (2) nicotine gum, such as Nicorette® (GlaxoSmithKline); (3) nicotine nasal spray, such as Nicotrol NS® (Pharmacia Consumer Healthcare); and (4) nicotine inhaler (Nicotrol® nicotine inhalation system (Pharmacia Consumer Healthcare).

Antidepressants have also been developed or proposed as therapy for smoking cessation. One of such antidepressants is bupropion. Bupropion HCl is available as antidepressant under the trade names Wellbutrin® and Wellbutrin SR® (GlaxoSmithKline). A sustained release formulation of bupropion HCl has been approved in the United States and several other countries as a therapy for smoking cessation and is marketed under the trade name Zyban® by GlaxoSmithKline. Zyban® can be used either alone or in combination with a nicotine transdermal system. Other antidepressants proposed for smoking cessation treatment include doxepin, imipramine (Nunn-Thompson et al., 1989 Clin. Pharm. 8: 710-720), and desipramine (Diana et al., 1990 Am. J. Physiol. 259: H1718-H1729).

Anxiolytics have also been explored or proposed as therapy for smoking cessation, which include, for example, isovaleramide (Balandrin et al., WO 94/28888), diazepam, meprobamate, metoprolol, ondansetron, and oxprenolol. (See also: Hughes J R; Stead L F; Lancaster T: Anxiolytics for Smoking Cessation. Cochrane Database of Systematic Reviews. Issue 1, 2002).

Another class of agents that has been explored as therapy for smoking cessation is nicotine receptor antagonists, examples of which include mecamylamine (Tennant et al., 1984 NIDA Res. Monogr. 55: 291-297), hexamethonium (Wotring et al., 1995 Neuroscience 67: 293-300), dihydro-beta-erythroidine (Stolerman et al., 1997 Psychopharmacology 129: 390-397), d-tubocurarine (Wotring et al., 1995), pempidine (Rapier et al., 1990 J. Neurochem. 54: 937-945), chlorisondamine (Caggiula et al., 1995 Psychopharmacology 122: 301-306), erysodine (Decker et al., 1995 Eur. J. Pharmacol. 280: 79-80) and trimethaphan camsylate (Hisayama et al., 1988 Br. J. Pharmacol. 95:465-472). Mecamylamine has been marketed as the anti-hypertensive agent under the tradename Inversine®, which is mecamylamine hydrochloride (Pfister, U.S. Pat. No. 2,831,027).

Still another class of agents that has been proposed as therapy for smoking cessation is opioid antagonists. (U.S. Pat. No. 6,004,970) Examples of such agents include naltrexone (also know as 17-(cyclopropylmethyl)-4,5-epoxy-3,14-dihydroxymorphinan-6-one), naloxone (also known as 4,5-epoxy-3,14-dihydroxy-17-(2-prophenyl)morphinan-6-one), and nalmefene (also known as 5alpha-17-(cyclopropylmethyl)-4,5-epoxy-6-methylenemorphinan-3,14-diol).

Combination therapies have also been proposed or developed for smoking cessation wherein two or more different therapeutic agents are co-administered to the patient. Examples of such combination therapies include (1) an antidepressant in combination with an anxiolytic (Glazer, U.S. Pat. No. 4,788,189); (2) nicotine receptor antagonist, such as mecamylamine, in combination with an anti-depressant, such as bupropion and doxepin, or an anxiolytic (U.S. Pat. No. 6,197,827), or in combination with a nicotine transdermal system (U.S. Pat. Nos. 5,574,052 and 5,316,759); (3) an opioid antagonist, such as nalmefene, naloxone, or naltrexone, in combination with nicotine, an antidepressant, or an anxiolytic (U.S. Pat. No. 6,004,970); and (4) D1/D5 antagonist or D1/D5 partial agonist in combination with buspirone or buproprion (U.S. Pat. No. 6,262,049).

Among the pharmacotherapies proposed or developed for smoking cessation mentioned above, none of them have shown to be satisfactory. For example, it was reported that the success rate for different nicotine replacement therapies ranges only 6%-16% at 12 months. A comparable low success rate was reported for bupropion hydrochloride. (Kolawole S. Okuyemi, MD, MPH; Jasjit S. Ahluwalia, MD, MPH, MS; Kari J. Harris, PhD, MPH. Pharmacotherapy of Smoking Cessation. Arch Fam Med. 2000;9:270-281). The effectiveness of many other proposed therapies have not been demonstrated, which is particularly true with anxiolytics as is observed by the authors of a recent article published in Cochrane Database of Systematic Reviews (Hughes J R; Stead L F; Lancaster T: Anxiolytics for smoking cessation. Cochrane Database of Systematic Reviews. Issue 1, 2002). In the aforementioned article, the authored reviewed the data of several clinical trials with the anxiolytics diazepam, meprobamate, metoprolol and oxprenolol for smoking cessation, and conclude that there is no consistent evidence that anxiolytics aid smoking cessation. Therefore, there clearly exists a continuing medical need for new pharmacologic therapies that will facilitate smoking cessation.

Reboxetine is shown to have antidepressant effect. However, unlike other known antidepressants, such as tricyclic antidepressants, which are norepinephrine reuptake inhibitors with varying levels of serotonin reuptake inhibtion and receptor blockade, or bupropion, which is a dopaminergic reuptake inhibitor, or fluoxetine, which is a serotonin reuptake inhibitor, reboxetine is shown to be the first potent selective, and specific norepinephrine reuptake inhibitor. U.S. Pat. No. 6,352,986 discloses a method of treating or enhancing the treatment of addictive disorders including tobacco addiction or nicotine addiction with reboxetine or derivatives or pharmaceutically acceptable salts thereof. Applicants now disclose a combination therapy with reboxetine and other smoking cessation enhancing agents that act synergistically to help a smoker quit smoking.

### SUMMARY OF INVENTION

It is an object of the present invention to provide a new therapy for smoking cessation in individuals who wish to quit or decrease smoking tobacco or the use of any tobacco product. It is also an object of the invention to provide a method for the treatment and/or relief of nicotine craving and/or smoking withdrawal symptoms in individuals who wish to quit or decrease the habit of smoking tobacco or the use of any tobacco product. It is a further object of the present invention to provide a novel composition for administration as a single therapy for promoting smoking cessation. These and other objects will become readily apparent to those skilled in the art as from the detailed description which follows.

One aspect of the present invention relates to a method of promoting smoking cessation in a human comprising administration of an effective amount of reboxetine or a pharmaceutically acceptable salt thereof in combination with administration of an effective amount of a smoking-cessation enhancing agent. The effective amount of reboxetine or a pharmaceutically acceptable salt thereof is typically from about 0.1 mg/day to about 20 mg/day. Reboxetine or a pharmaceutically acceptable salt thereof and the second agent can be administered either separately or together in a single composition.

Another aspect of the present invention relates to a composition for administration to a human for promoting smoking cessation comprising a therapeutically effective amount of reboxetine or a pharmaceutically acceptable salt thereof in combination with a pharmaceutically effective amount of a smoking-cessation enhancing agent. The pharmaceutical compositions of the invention can be prepared for oral, parenteral, rectal, transdermal, or buccal administration, or in a form suitable for administration by inhalation or intranasal administration.

Specific smoking-cessation enhancing agents for the present invention include nicotine, antidepressants other than reboxetine, anxiolytics, nicotine receptor antagonists, or opioid antagonists.

### DETAILED DESCRIPTION OF THE INVENTION

The full disclosure of all the patents, patent applications, and publications cited in the present application is incorporated herein by reference.

Reboxetine is the generic name for 2-[α(2-ethoxyphenoxy)benzyl)-morpholine. As used herein, the term “reboxetine” refers to the RR-enantiomer, SS-enantiomer, racemic mixture, or pharmaceutically acceptable salts, of 2-[α(2-ethoxyphenoxy)benzyl]-morpholine. Examples of suitable pharmaceutical salts of reboxetine for the present invention include reboxetine methanesulfonate (also called reboxetine mesylate), reboxetine fumarate and reboxetine succinate.

Reboxetine and methods of preparation of the racemic mixture of reboxetine are described in U.S. Pat. Nos. 4,229,449, 5,068,433, and 5,391,735. Individual stereoisomers of reboxetine can be obtained by resolution of the racemic mixture of enantiomers using conventional methods generally known by those skilled in the art. Such methods include, but are not limited to, resolution by simple crystallization and chromatographic techniques, for example, as set forth in GB 2,167,407. Other methods of preparation are described in U.S. Pat. No. 5,068,433 and U.S. Pat. No. 5,391,735. Pharmaceutical compositions and methods of administration of reboxetine are also described in U.S. Pat. No. 4,229,449. Reboxetine is also commercially available under the tradename Edronax®.

In one aspect, the invention provides a method for promoting smoking cessation comprising administration to a human in need thereof of an effective amount of reboxetine and a smoking-cessation enhancing agent. A smoking-cessation enhancing agent can be an agent that, when administered alone, has smoking-cessation promoting effect, or can be an agent that has no smoking-cessation promoting effect by itself but can enhance the smoking-cessation promoting effect of reboxetine when co-administered with reboxetine.

The smoking-cessation enhancing agents contemplated in for use in the present invention includes, but not limited to, nicotine, antidepressants, anxiolytics, nicotine receptor antagonists, and opioid receptor antagonists.

Reboxetine of the invention can be administered by any suitable means, such as local or systemic administration. Systemic administration can be via any suitable method known in the art such as, for example, oral administration of lozenges, tablets, capsules, granules, or other oral dosage forms; intramuscular, intradermal, or intravenous administration, such as by sterile injections; and transdermal administration, such as transdermal patch. A particular convenient method is oral dosing once or twice a day. More than twice daily administrations (e.g., 3, 4, 5 or 6 administrations per day) are also expressly contemplated herein.

Reboxetine can be administered in a wide dose range of active ingredient from about 0.1 mg to about 20 mg per patient per day. The exact dose levels may vary depending on a variety of factors such as stereochemical character of the reboxetine used, nature and dose of the second agent used, the formulation and route of administration of each agent, and the condition of the patient and the severity of the conditions to be treated. Generally, when the S,S-enantiomer of reboxetine is used, the daily dose range is from about 0.1 to about 5 mg per patient, typically from about 0.2 to about 2 mg per patient, such as 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 mg, or even from about 0.5 to about 1 mg per patient, such as 0.5, 0.6, 0.7, 0.8, 0.9, or 1.0 mg. In the case where racemic reboxetine is to be administered, the daily dose is generally from about 0.1 mg to about 10 mg, but cant be 0.2 to 4 mg per patient, or even 0.3 to 2 mg per patient, such as 0.3, 0.4, 0.5, 0.6, 0.7, 0.8, 0.9, 1.0, 1.1, 1.2, 1.3, 1.4, 1.5, 1.6, 1.7, 1.8, 1.9, or 3.0. The ideal dosing would be routinely determined by an evaluation of clinical trials and the needs of the patient.

In one embodiment, the invention provides a method for promoting smoking cessation comprising administration of an effective amount of reboxetine in combination with administration of an effective amount of a smoking-cessation enhancing agent wherein the smoking-cessation enhancing agent is nicotine. Nicotine in the present invention may be administered by any conventional means, such as, for example, using a transdermal nicotine delivery system (e.g. skin patch), nicotine chewing gum, nicotine inhaler, or other nicotine delivery systems that are used in the nicotine replacement therapies. Nicotine replacement therapies are known in the art and products for such therapies are available on the market, which products include, but are not limited to, nicotine transdermal patchs, such as NicoDerm® CQ® (GlaxoSmithKline), Habitrol® (Novartis Consumer Health), and Nicotrol® (Pharmacia Consumer Healthcare); nicotine gum, such as Nicorette® (GlaxoSmithKline), that delivers nicotine in either 2 or 4 mg doses through buccal (mouth) absorption; nicotine nasal spray, such as Nicotrol NS® (Pharmacia Consumer Healthcare); and nicotine inhaler (Nicotrol® nicotine inhalation system (Pharmacia Consumer Healthcare). Information about the dosages and administration for each of these products can be found in the product insert for each product. Generally, nicotine is administered in an amount of from about 1 mg to about 100 mg, preferably from about 3 mg to about 75 mg, more preferably from about 5 mg to about 50 mg. The transdermal doses of nicotine typically range from about 5 mg to about 42 mg, but preferably from about 5 mg to about 21 mg.

In another embodiment, the invention provides a method for promoting smoking cessation comprising administration of an effective amount of reboxetine in combination with administration of an effective amount of a smoking-cessation enhancing agent wherein the smoking-cessation enhancing agent is an antidepressant. By “antidepressant” it is meant a therapeutic agent that is useful for the treatment of depressions in humans. Examples of specific antidepressants of the present invention include bupropion, doxepin, or a pharmaceutically acceptable salt of the above agents.

Bupropion is the generic name for 1-(3-chlorophenyl)-2-[(1,1-dimethylethyl)amino]-1-propanone. The preparation of bupropion and its pharmaceutically acceptable salts is disclosed in U.S. Pat. Nos. 3,819,706 and 3,885,046. A transdermal delivery system of bupropion is disclosed in U.S. Pat. No. 6,312,716. A particular salt of bupropion, bupropion hydrochloride (bupoprion HCl), is preferred in the present invention. Formulations of bupropion hydrochloride that may be used in the invention include those that are commercially available under tradenames of Wellbatrin® , Wellbutrin®, and Zyban®. Information about the dosage and administration of these products can be found in the product insert. The doses of bupropion hydrochloride for use in the present invention is generally from about 50 mg to about 400 mg per day, and preferably from about 150 mg to about 300 mg per day.

Doxepin is the generic name for 1-propanamine, 3-dibenz[b,e]oxepin-11 (6 H)ylidene-N,N-dimethyl. The preparation, pharmaceutical composition, and use of doxepin are disclosed in U.S. Pat. Nos. 3,438,981 and 3,420,851. A particular salt of doxepin, doxepin hydrochloride, is preferred in the present invention. Doxepin hydrochloride is available on the market under tradenames Adapin®, Aponal®, Curatin®, Novoxapin®, Quitaxon®, and Sinequan®. Doxepin can be administered by any suitable means, preferably orally with the dosage form of, for example, tablets or capsules. The dosage of doxepin hydrochloride in the present invention is generally from 25 mg/day to 300 mg/day. For most patients with mild to moderate tobacco dependence the usual optimum dose range is 75 mg/day to 150 mg/day. The total daily dosage of doxepin hydrochloride may be given on a divided or once-a-day dosage schedule.

Additional antidepressants contemplated for the present invention include, but not limited to, amitriptyline (100-30 mg per day), clomipramine (200-250 mg per day), desipramine (100-300 mg per day), imipramine (100-300 mg per day), nortriptyline (50-200 mg per day), protriptyline (20-60 mg per day), trimipramine (100-300 mg per day), fluoxetine (10-80 mg per day), fluvoxamine (100-300 mg per day), paroxetine (20-50 mg per day), sertraline (50-200 mg per day), phenelzine (45-90 mg per day), tranylcypromine (20-50 mg per day), amoxapine (200-600 mg per day), maprotiline (150-200 mg per day), trazodone (200-600 mg per day), tomoxetine (40-80 mg per day), duloxetine (40-80 mg per day) nefazodone (300-600 mg per day), venlafaxine (75-375 mg per day), and mirtazapine (15-45 mg per day); and their pharmaceutically acceptable salts and optical isomers. The preferred dosage ranges for any one of the above listed anti-depressants in the present invention would likely lie in the low to mid-range dosages suggested for each agent.

In yet another embodiment, the invention provides a method for promoting smoking cessation comprising administration of an effective amount of reboxetine in combination with administration of an effective amount of a smoking-cessation enhancing agent wherein the smoking-cessation enhancing agent is an anxiolytic. By “anxiolytic” it is meant a therapeutic agent that is useful for the treatment of anxiety in humans. Examples of specific anxiolytics include benzodiazepines, triazolobenzodiazepines, and non-benzodiazepine anxiolytics such as buspirone HCl.

Benzodiazepine anxiolytics are well known in the art. Examples of benzodiazepines suitable for the present invention include alprazolam (available under the tradename Xanax), chlordiazepoxide (available under the tradename Librium), clorazepate (available under the tradename Tranxene), diazepam (available under the tradename Valium), halazepam (available under the tradename Paxipam), lorazepam (available under the tradename Ativan), oxazepam (available under the tradename Serax), prazepam (available under the tradename Centrax), midazolam (available under the tradename Versed), and clonazepam (available under the tradename Kionopin). Suitable dosage range of each of the above benzodiazepine anxiolytics for use in the present invention generally lie in the low to mid-range of their respective recommended dosage ranges for treating anxiety. The dose frequency and route of administration for each of the above benzodiazepines anxiolytics for treating anxiety are also suitable for promoting smoking cessation in the present invention.

One example of non-benzodiapine anxiolytics suitable in the present invention is buspirone or a pharmaceutically acceptable salt thereof, such as buspirone HCI. U.S. Pat. Nos. 3,717,634 and 4,182,763 describe the synthesis, pharmaceutical composition and use of buspirone as an anxiolytic. Its synthesis is described in U.S. Pat. No. 3,717,634. Buspirone HCI is commercially available under such tradenames as Bespar®, BuSpar®, Buspinol®, Busirone®, Censpar®, Lucelan® and Travine®. The dosage range of buspirone HCl for the present invention is can be from about 1 to 50 mg per day, typically from about 3 to 25 mg per day, administered orally as divided doses three times a day. Additional anxiolytics for use in the present invention include hydroxyzine (50-400 mg per day) and meprobamate (400-1600 mg per day).

In still another embodiment, the invention provides method of promoting smoking cessation comprising administration of an effective amount of reboxetine in combination with an effective amount of a smoking-cessation enhancing agent wherein the a smoking-cessation enhancing agent is a nicotine receptor antagonist. An example of suitable nicotine receptor antagonists in the present invention is mecamylamine (also known as 3-methylamino-2,2,3-trimethylnorcamphane) or a pharmaceutically acceptable salt thereof, such as mecamylamine HCl. U.S. Pat. No. 2,831,027 describes the synthesis of mecamylamine. A tablet formulation of mecamylamine HCl is available under the tradename Inversine® (Layton) as an oral antihypertension agent and ganglion blocker. Other nicotinic antagonists contemplated for use in the present invention include dihydro-beta-erythroidine; tubocurarine chloride; d-tubocurarine; amantadine; pempidine; erysodine; chlorisondamine; hexamethonium; and trimethaphan camsylate. The pharmaceutical salts of these compounds are also contemplated for use in the present invention. The nicotine receptor antagonist can be administered by any suitable means known in the art. The effective amount of a nicotine receptor antagonist to be administered in the present invention varies depending on various factors, such as the particular compound used, but generally ranges from about 0.1 mg/day to about 400 mg/day. For most patients with mild to moderate tobacco dependence, the usual optimum dose range is from 1 mg/day to 150 mg/day. In some embodiments mecamylamine HCl is administered as an oral tablet, at doses ranging generally from 2 mg/day to 75 mg/day.

In yet another embodiment, the invention provides method of promoting smoking cessation comprising administration of an effective amount of reboxetine in combination with an effective amount of a smoking-cessation enhancing agent wherein the a smoking-cessation enhancing agent is an opioid antagonist. Any opioid antagonist may be employed. Examples of the opioid antagonists in the present invention include naltrexone and other structurally related opiate antagonists such as naloxone, nalmefene, and mixtures thereof, with naltrexone being preferred. Opioid antagonists can be administered locally or systemically. In some embodiments, the opioid antagonist is administered orally. The effective amount of oral naltrexone in the present invention is generally from about 10 mg to about 150 mg per day, and is typically from about 15 mg to about 75 mg per day for most patients with moderate tobacco dependence.

The precise amount of any compounds or its pharmaceutically acceptable salt to be administered to a patent in the present invention is not fixed, but is dependent on numerous factors known to one skilled in the art, such as the particular compound selected, the age, weight, and general condition of the patient, severity of the tobacco dependence, whether other compound is administered, the particular dosage form used, route of administration, and so forth, but may easily be determined by routine experimentation. As a general guidance, therapy should start with a low dose. This initial dosage should be gradually increased or decreased at intervals generally not less than 2 days until the desired response occurs, taking into account the adverse effects of the agents.

Reboxetine and the smoking-cessation enhancing agent can be administered separately, either simultaneously or sequentially in any order at different points in time. However, if not administered simultaneously, they should be administered in such a fashion as to provide the desired treatment effect resulting from effective simultaneous blood levels of both agents. Suitable dosing intervals and dosing order with such components will be readily apparent to those skilled in the art, once armed with the present disclosure. Alternatively, reboxetine and the smoking-cessation enhancing agent can be administered in a single pharmaceutical composition.

Thus, in another aspect, the present invention provides a pharmaceutical composition for administration to a human in need thereof for promoting smoking cessation, which composition comprises an effective amount of reboxetine and an effective amount of a smoking-cessation enhancing agent.

In one embodiment, the smoking-cessation enhancing agent is an antidepressant. Examples of specific antidepressants of the present invention include bupropion, doxepin, amitriptyline, clomipramine, desipramine, imipramine, nortriptyline, protriptyline, trimipramine, fluoxetine, fluvoxamine, paroxetine, sertraline, phenelzine, tranylcypromine, amoxapine, maprotiline, trazodone, tomoxetine, nefazodone, venlafaxine, and mirtazapine, and their pharmaceutically acceptable salts and optical isomers.

In another embodiment, the smoking-cessation enhancing agent is an anxiolytic. Examples of specific anxiolytics suitable for the present invention include benzodiazepines, such as alprazolam, chlordiazepoxide, clorazepate, diazepam, halazepam, lorazepam, oxazepam, prazepam, midazolam, and clonazepam. Examples of non-benzodiazepine anxiolytics suitable for the present invention include buspirone HCl, hydroxyzine, and meprobamate.

In yet another embodiment, the smoking-cessation enhancing agent is nicotine.

In still another embodiment, the smoking-cessation enhancing agent is a nicotine receptor antagonist. Example of suitable nicotine receptor antagonists in the present invention include mecamylamine, dihydro-beta-erythroidine, tubocurarine chloride, d-tubocurarine, amantadine, pempidine, erysodine, chlorisondamine, hexamethonium, trimethaphan camsylate, and pharmaceutically acceptable salts thereof.

In yet another embodiment, the smoking-cessation enhancing agent is an opioid antagonist. Examples of the opioid antagonists in the present invention include naltrexone and other structurally related opiate antagonists such as naloxone, nalmefene, and mixtures thereof, with naltrexone being preferred.

Desirably, the daily dose of reboxetine, when the (S′S) enantiomer is used, contains from about 0.1 mg to about 2.0 mg. More preferably, each dose of the reboxetine contains about 0.2 to about 0.8 mg of the (S′S) enantiomer, and even more preferably, each dose contains from 0.05 to about 1 mg of the (S′S) enantiomer This dosage form permits the full daily dosage to be administered in one or two oral doses. to This will allow for final formulations containing 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0 mg of (S′S) enantiomer.

In the case racemic reboxetine is to be administered, the daily dose of reboxetine contains from about 0.1 mg to about 4.0 mg. More preferably, each dose of the reboxetine contains about 0.2 to about 3.0 mg, and even more preferably, contains about 0.1 to about 2 mg of racemic reboxetine. This dosage form permits the full daily dosage to be administered in one or two oral doses. This will allow for final formulations containing 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8.2.9, 3.0, 3.1, 3.2, 3.3, 3.4, 3.5, 3.6, 3.7, 3.8, 3.9, or 4.0 mg of racemic reboxetine.

The pharmaceutical compositions of the invention can be prepared for oral, buccal, parenteral, rectal, transdermal or transmuccosal administration, or in a form suitable for administration by inhalation or intranasal administration. The compositions of the invention can be formulated by suitable technologies known to a person skilled in the art.

For oral administration, the pharmaceutical compositions of the invention may take the form of, for example, tablets or capsules prepared by conventional means with pharmaceutically acceptable excipients such as binding agents (e.g., pregelatinised maize starch, polyvinylpyrrolidone or hydroxypropyl methylcellulose);

fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate); glidants; artificial and natural flavors and sweeteners; artificial or natural colors and dyes; and solubilizers. The pharmaceutical compositions of the invention for oral administration may also take the form of liquid preparations, such as solutions, syrups or suspensions, or they may be presented as a dry product for reconstitution with water or other suitable vehicles before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

For buccal administration, the composition may take the form of tablets or lozenges formulated in conventional manners.

For parenteral administration by injection, the compositions may take such forms as suspensions, solutions or emulsions in oily or aqueous vehicles, and may contain formulating agents such as suspending, stabilizing and/or dispersing agents. Alternatively, the active ingredients may be in powder form for reconstitution with a suitable vehicle, e.g., sterile pyrogen-free water, before use.

For rectal administration the pharmaceutical compositions of the invention may take the form of suppositories or retention enemas, e.g., containing conventional suppository bases such as cocoa butter or other glycerides.

For transdermal administration, the composition of the invention can be formulated in the form of a transdermal patch. The patch may comprise a reservoir containing the active agents of the invention and means for applying the reservoir in drug-transmitting relation to the skin or a membrane of a patient. The reservoir may be adapted to be placed in direct contact with the skin or membrane, or a rate-controlling membrane may be interposed between the reservoir and the skin or membrane. The reservoir may contain the active agents of the invention in liquid form or as a solution, or contain a solid or semi-solid polymer matrix having the active agents of the invention dispersed or dissolved therein. The reservoir may further include a skin permeation enhancing agent that is adapted to be co-delivered with the active agents of the invention. The patch may further comprise an impermeable backing layer which overlays or envelops the reservoir remote from the skin or membrane, and an adhesive layer may be provided around the reservoir or between the reservoir and the skin for securing the patch to a patient.

For intranasal administration or administration by inhalation the pharmaceutical compositions of the invention can be formulated in the form of a solution or suspension which can be delivered from a pump spray container that is squeezed or pumped by the patient, or as an aerosol spray presentation from a pressurized container or nebulizer, with the use of a suitable propellant (e.g., dichlorodifluoromethane, trichlorofluoromethane, dichlorotetrafluoroethane, carbon dioxide or other suitable gas). In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of an active compound and a suitable powder base such as lactose or starch.

### Definitions

The term “promoting smoking cessation” as used in this application refers to helping a human to quit or reduce tobacco smoking or to quit or reduce use of tobacco products; to decrease craving for tobacco products or nicotine; to reduce relapse to heavy smoking during detoxification or once smoking abstinence has been achieved; or to alleviate various symptoms of smoking withdrawal syndromes.

The term “smoking-cessation enhancing agent” as used in this application refers to a therapeutic agent, compound, or composition, other than reboxetine, the co-administration of which with reboxetine provides therapeutic synergy in promoting smoking cessation. By “therapeutic synergy” in promoting smoking cessation is meant an efficacy in promoting smoking cessation that is greater than the efficacy that would be observed upon administration of either reboxetine or the smoking-cessation enhancing agent.

The term “effective amount” of reboxetine and a smoking-cessation enhancing agent is an amount which, when co-administered to the patient, is sufficient to provide therapeutic synergy in promoting smoking cessation.

The terms “craving for tobacco products or nicotine” and “smoking withdrawal symptoms” as used herein both refer to any physical or psychological reaction relating to breaking the habit of smoking tobacco or using any tobacco product or decreasing the frequency or intensity of smoking tobacco or using any tobacco product.

The term “pharmaceutically acceptable” is used herein to describe materials that are non-toxic at the amount used and suitable for administration to humans.

### EXAMPLES

Without further description, it is believed that one of ordinary skill in the art, using the preceding description and the following illustrative examples, can make and utilize the compounds of the present invention and practice the claimed methods.

### Example 1

A composition is prepared by combining about 0.2 mg reboxetine in either its racemic or +(S,S) entantiomer form with about 50 mg bupropion in a pharmaceutically acceptable carrier in a single tablet or capsule and is administered orally at a dose frequency between one to six tablets daily.

### Example 2

A formulation comprising about 1.0 mg reboxetine and about 150 mg bupropion is combined into a single tablet or capsule and is administered orally at a dose frequency between one to six tablets daily.

### Example 3

A formulation comprising about 1.5 mg reboxetine and about 50 mg bupropion is combined into a single tablet or capsule and is administered orally at a dose frequency between one to six tablets daily.

### Example 4

A formulation comprising about 1.5 mg reboxetine and about 150 mg bupropion is combined into a single tablet or capsule and is administered orally at a dose frequency between one to six tablets daily.

### Example 5

A 0.8 0 mg tablet of reboxetine is taken orally two times daily, and a 150 mg tablet of bupropion is taken two times daily, in the morning and evening.

What is claimed is:

1. A method of promoting smoking cessation in a human comprising administering to the human an effective amount of reboxetine in combination with administration of an effective amount of a smoking-cessation enhancing agent.

2. The method of claim 1, wherein the reboxetine is in the form of racemic mixture of the S,S- and R,R-enantiomers, or pharmaceutically acceptable salts thereof, in any ratio.

3. The method of claim 1, wherein the reboxetine is in the form of pure or substantially pure S,S-enantiomer or pharmaceutically acceptable salts thereof.

4. The method of claim 2, wherein the effective amount of reboxetine is from about 0.1 mg to about 20 mg per patient per day.

5. The method of claim 2, wherein the effective amount of reboxetine is from about 0.1 mg to about 10 mg per patient per day.

6. The method of claim 2, wherein the effective amount of reboxetine is from about 0.2 mg to about 5 mg per patient per day.

7. The method of claim 2, wherein the effective amount of reboxetine is from about 0.3 mg to about 3 mg per patient per day.

8. The method of claim 3, wherein the effective amount of reboxetine is from about 0.1 mg to about 6 mg per patient per day.

9. The method of claim 3, wherein the effective amount of reboxetine is from about 0.2 mg to about 4 mg per patient per day.

10. The method of claim 3, wherein the effective amount of reboxetine is from about 0.3 mg to about 3 mg per patient per day.

11. The method of claim 1 wherein the reboxetine and the smoking-cessation enhancing agent are administered separately.

12. The method of claim 1 wherein the reboxetine and the smoking-cessation enhancing agent are administered in a single composition.

13. The method of claim 1, wherein the smoking-cessation enhancing agent is nicotine, an antidepressant, an anxiolytic, a nicotine receptor antagonist, an opioid antagonist, or the mixture thereof.

14. The method of claim 1 wherein smoking-cessation enhancing agent is nicotine.

15. The method of claim 14 wherein the nicotine is in a dosage form of transdermal patch, chewing gum, lozenge, capsule, tablet, inhalant, or nasal spray.

16. The method of claim 1 wherein the smoking-cessation enhancing agent is an antidepressant.

17. The method of claim 16 wherein the antidepressant is selected from the group consisting of buproion, doxepin, amitriptyline, clomipramine, desipramine, imipramine, nortriptyline, protriptyline, trimipramine, fluoxetine, fluvoxamine, paroxetine, sertraline, phenelzine, tranylcypromine, amoxapine, maprotiline, tomoxetine, duloxetine, trazodone, nefazodone, venlafaxine, mirtazapine, and pharmaceutically acceptable salts of any said antidepressant.

18. The method of claim 16 wherein the antidepressant is doxepin or a pharmaceutically acceptable salt thereof.

19. The method of claim 16 wherein the antidepressant is buproion or a pharmaceutically acceptable salt thereof.

20. The method of claim 1 wherein smoking-cessation enhancing agent is an anxiolytic.

21. The method of claim 20 wherein the anxiolytic is a benzodiazepine.

22. The method of claim 20 wherein the anxiolytic is alprazolam, chlordiazepoxide, clorazepate, diazepam, halazepam, lorazepam, oxazepam, prazepam, midazolam, clonazepam, or pharmaceutically acceptable salts thereof.

23. The method of claim 20 wherein the anxiolytic is buspirone, hydroxyzine, meprobamate, or pharmaceutically acceptable salts thereof.

24. The method of claim 20 wherein the anxiolytic is buspirone HCl.

25. The method of claim 1 wherein smoking-cessation enhancing agent is a nicotine receptor antagonist.

26. The method of claim 25 wherein the nicotine receptor antagonist is selected from mecamylamine, dihydro-beta-erythroidine, tubocurarine chloride, d-tubocurarine, amantadine, pempidine, erysodine, chlorisondamine, hexamethonium, trimethaphan camsylate, and a pharmaceutically acceptable salt of any said nicotine receptor antagonist.

27. The method of claim 25 wherein the nicotine receptor antagonist is mecamylamine or a pharmaceutically acceptable salt thereof.

28. The method of claim 1 wherein the smoking-cessation enhancing agent is an opioid antagonist.

29. The method of claim 28 wherein the opioid antagonist is naltrexone, naloxone, nalmefene, or a pharmaceutically acceptable salt of any said opioid antagonist.

30. The method of claim 28 wherein the opioid antagonist is naltrexone or a pharmaceutically acceptable salt thereof.

31. A pharmaceutical composition for administration to a human for promoting smoking cessation, said composition comprising an effective amount of reboxetine and an effective amount of a smoking-cessation enhancing agent.

32. The pharmaceutical composition of claim 31 wherein the smoking-cessation enhancing agent is nicotine, an antidepressant, an anxiolytic, an nicotine receptor antagonist, an opioid antagonist, or the mixture thereof.

33. The pharmaceutical composition of claim 31 wherein the smoking-cessation enhancing agent is nicotine.

34. The pharmaceutical composition of claim 31 wherein the smoking-cessation enhancing agent is an antidepressant.

35. The pharmaceutical composition of claim 34 wherein the antidepressant is selected from the group consisting of buproion, doxepin, amitriptyline, clomipramine, desipramine, imipramine, nortriptyline, protriptyline, trimipramine, fluoxetine, fluvoxamine, paroxetine, sertraline, phenelzine, tranylcypromine, amoxapine, tomoxetine, duloxetine, maprotiline, trazodone, nefazodone, venlafaxine, mirtazapine, and pharmaceutically acceptable salts of any said antidepressant.

36. The pharmaceutical composition of claim 34 wherein the antidepressant is doxepin or a pharmaceutically acceptable salt thereof.

37. The pharmaceutical composition of claim 34 wherein the antidepressant is buproion or a pharmaceutically acceptable salt thereof.

38. The pharmaceutical composition of claim 31 wherein smoking-cessation enhancing agent is an anxiolytic.

39. The pharmaceutical composition of claim 38 wherein the anxiolytic is a benzodiazepine.

40. The pharmaceutical composition of claim 38 wherein the anxiolytic is alprazolam, chlordiazepoxide, clorazepate, diazepam, halazepam, lorazepam, oxazepam, prazepam, midazolam, clonazepam, or pharmaceutically acceptable salts thereof.

41. The pharmaceutical composition of claim 38 wherein the anxiolytic is buspirone, hydroxyzine, meprobamate, or pharmaceutically acceptable salts thereof.

42. The pharmaceutical composition of claim 38 wherein the anxiolytic is buspirone HCl.

43. The pharmaceutical composition of claim 1 wherein smoking-cessation enhancing agent is a nicotine receptor antagonist.

44. The pharmaceutical composition of claim 43 wherein the nicotine receptor antagonist is selected from mecamylamine, dihydro-beta-erythroidine, tubocurarine chloride, d-tubocurarine, amantadine, pempidine, erysodine, chlorisondamine, hexamethonium, trimethaphan camsylate, and a pharmaceutically acceptable salt of any said nicotine receptor antagonist.

45. The pharmaceutical composition of claim 43 wherein the nicotine receptor antagonist is mecamylamine or a pharmaceutically acceptable salt thereof.

46. The pharmaceutical composition of claim 1 wherein the smoking-cessation enhancing agent is an opioid antagonist.

47. The pharmaceutical composition of claim 46 wherein the opioid antagonist is naltrexone, naloxone, nalmefene, or a pharmaceutically acceptable salt of any said opioid antagonist.

48. The pharmaceutical composition of claim 46 wherein the opioid antagonist is naltrexone or a pharmaceutically acceptable salt thereof.

49. The method of claim 19 wherein the effective amount of bupropion or a pharmaceutically acceptable salt thereof is from about 40 mg to about 250 mg per day.

50. The method of claim 19, wherein the effective amount of bupropion or a pharmaceutically acceptable salt thereof is from about 50 mg to about 200 mg per day.

51. The method of claim 19, wherein the effective amount of bupropion or a pharmaceutically acceptable salt thereof is from about 75 mg to about 150 mg.

52. The method of claim 14 wherein the effective amount of nicotine is from about 7 mg to about 42 mg per day.

53. The method of claim 14 wherein the effective amount of nicotine is from about 3 mg to about 21 mg per day.

54. The composition of claim 31, wherein the reboxetine is in the form of racemic mixture of the S,S- and R,R-enantiomers, or pharmaceutically acceptable salts thereof, in any ratio.

55. The composition of claim 31, wherein the reboxetine is in the form of pure or substantially pure S,S-enantiomer or pharmaceutically acceptable salts thereof.
